(19) United States
(12) Patent Application Publication
(10) Pub. No.: US 2008/0064922 A1
(43) Pub. Date: Mar. 13, 2008

(54) Color Image Pickup And Display Device

(75) Inventors: Tatsuo Uchida, Sendai-shi (JP); Yoshito Suzuki, Sayama-shi (JP); Go Sugiura, Tokyo (JP); Yukio Taniguchi, Tokyo (JP)

Correspondence Address: DARBY & DARBY P.C., P.O. BOX 770, Church Street Station, New York, NY, 10008-0770, US

(73) Assignees: PENTAX CORPORATION, Itabashi-ku, Tokyo (JP); TOHOKU UNIVERSITY, Sendai-shi, Miyagi (JP); SOYOU CORPORATION, Sayama-shi, Saitama (JP)

(21) Appl. No.: 11/576,117

(22) PCT Filed: Nov. 10, 2005
(86) PCT No.: PCT/JP05/21074

§ 371 Date: Mar. 27, 2007

(30) Foreign Application Priority Data
Nov. 11, 2004 (JP) 2004-328258

(51) Int. Cl. A61B 1/04; H04N 9/04

(52) U.S. Cl. 600/109; 348/266; 348/E09.002

(57) ABSTRACT

A color image pickup and display device that can be simply structured and provided at a low price. A lighting source irradiates lights of primary colors in a manner capable of being switched in a predetermined sequence. Monochrome solid-state image sensors are used as image pickup means, while a backlight type monochrome LCD element is used as a display of a display device. The LCD element can switch the color of backlight to the same color as that of the lighting source synchronously with the switching of color thereof. Thus, the backlight can emit light of the same color as that of the light emitted by the lighting source when the picture signals outputted by the sensors in response to the image observed by the light of the lighting source are displayed by the LCD element.

### CROSS-REFERENCE TO PRIOR APPLICATIONS

This is a U.S. national phase application under 35 U.S.C. §371 of International Patent Application No. PCT/JP2005/021074 filed Nov. 10, 2005, and claims the benefit of Japanese Application No. 2004-328258, filed Nov. 11, 2004. The International Application was published in English on May 18, 2006 as International Publication No. WO 2006/052011 under PCT Article 21(2) the content of which is incorporated herein in its entirety.

### TECHNICAL FIELD

The present invention relates to a color image pickup and display device, particularly to one in which illuminating light is irradiated to a subject so that light reflected therefrom is picked up to thereby display the color image of a subject thus picked up on a display of a display device in real time. Such color image pickup and display device is applicable to specific color image pickup and display devices such as endoscopes, airway establish instruments and the like that are suitable for visually inspecting a state inside a limited area, or inside a narrow and elongated space such as inside an endoscope or an airway establishment instrument.

### BACKGROUND ART

Endoscopes are used for diagnostic purpose, by inserting the same from the mouth into alimentary system such as esophagus or by inserting the same from the anus in the intestinal system such as large intestines, to visually grasp an internal situation therein.

Many conventional endoscopes have employed a solid-state image sensor with which monochrome images are picked up and then reproduced on a display to monitor the same. Recently, however, an increasing number of endoscopes are designed to pick up color images and then reproduce them on display. Some examples of those endoscopes are disclosed in Japanese Un-Examined Patent Publication Nos. 09-037236 and 06-331906.

According to the technique disclosed in Japanese Un-Examined Patent Publication No. 09-037236, solid-state image sensor for color imaging is used to output, from the solid-state image sensor, color image pickup signals, which are then processed by a color video processor, to thereby output color image signals from the vide processor so that they are reproduced on a color image monitor and observed.

On the other hand, according to the technique disclosed in Japanese Un-Examined Patent Publication No. 06-331906, solid-state image sensor for monochrome images is used, wherein illumination light of three primary colors: Red (R), Green (G) and Blue (B) is irradiated in sequence with a time lag so that an image of a subject is taken by the solid-state image sensor sequentially, corresponding to the respective primary colors, and then pickup image signals outputted from the solid-state image sensor are sequentially processed by color display processing device to produce color image signals so that they are able to be reproduced on a color monitor. Incidentally, monochrome images can also be reproduced and displayed according to this technique.

For instruments for urgent medical treatment, there is proposed an airway establishment apparatus in Japanese Un-Examined Patent Publication No. 2002-17862, for example. This instrument is to establish airway by inserting a main body of the instrument from the mouth as airway needs to be established prior to performing urgent medical treatment such as artificial respiration etc. due to abnormal cardiopulmonary function caused by injuries suffered in accidents or the like. Only those who have a specific medical license would be permitted to use such airway establishment apparatus in the past, due to the use thereof falling under the category of a medical act. According to revised law stressing the importance of emergency, emergency life guards also are now qualified to use the instrument, thus leading to remarkable effects that the ratio of persons whose lives were saved by the life guards' use of airway establishment instruments has increased dramatically.

Also, foreign body removal instruments for removing foreign bodies such as phlegm, rice cakes or the like by suctioning them are also becoming important in the medical treatment. In the case of phlegm removal instruments, for example, it will be unexpectedly difficult for patients who have declined in physical strength due to aging or long-term medical treatment to discharge phlegm from their body by coughing or the like. Therefore, it is not rare that they suffer from phlegm that is not capable of being discharged by themselves due to their insufficient coughing power when phlegm is always produced. Then, a phlegm removal instrument including a phlegm suctioning hose to be inserted in the vicinity of airway to suction phlegm has come into use. In addition, rice cakes sometimes choke people, particularly elderly people to death, who can be saved if a foreign body such as the rice cake is removed using the foreign body removal instrument.

According to the technique described in the above-mentioned Japanese Un-Examined Patent Publication No. 09-037236, it is, first of all, necessary to use a color solid-state image sensor as a solid-state image sensor, and thus there has been a problem that such color solid-state image sensors have made it difficult to obtain higher resolution using a small-sized sensor, since the resolution of a monochrome solid-state image sensor is more excellent when comparing a monochrome solid-state image sensor with a color solid-state image sensor of the same chip size as the former. This, in other words, means that a color solid-state image sensor needs to have a larger chip size than a monochrome solid-state image sensor if both have the same resolution.

This is because the color solid-state image sensor includes color separation filters (which are often complementary color filter(s), but sometimes primary color filter(s)) of different colors which are arranged at respective pixels in an image pickup area formed on a semiconductor substrate according to a predetermined array, such that one color is reproduced by signals based on signal charges from four pixels, for example.

Particularly, miniaturization of image pickup means is very important to endoscopes and the like when needs for lesser pain to be caused by insertion thereof and for narrower parts of a human (or animal) organ to be observed are to be met. According to the above-mentioned technique, however, it is difficult to meet those needs.

Secondly, according to Japanese Un-Examined Patent Publication No. 09-037236, picture signals obtained by the image pickup by the color solid-state image sensor need to be processed using a video processor of a complex circuitry so as to reproduce them on a color monitor, and thus there has been a problem that the price of the system becomes expensive.

Also, according to the technique disclosed in Japanese Un-Examined Patent Publication No. 06-331906, whilst a monochrome solid-state image sensor is used as a solid-state image sensor, illumination lights of primary colors: red (R), green (G) and blue (B) are irradiated in sequence with a time lag so that a image to be observed is picked up by each primary color, using the solid-state image sensor. Thus, it can meet the need for the small-sizing of the solid-state image sensor. In that sense, this technique can be said to have an advantage.

However, this technique also has a problem, because the picture signals of the respective different primary colors irradiated in sequence with a time lag are outputted from the monochrome solid-state image sensor and then they need to be converted into color picture signals using a video processor of a complex circuitry so as to reproduce them on a color monitor, and thus there has been a problem that the endoscope system becomes complex and expensive.

Moreover, the conventional airway establishment instrument disclosed in Japanese Un-Examined Patent Publication No. 2002-17862 also has had a problem that it is not easy to operate accurately on an emergency site where an accident occurred, despite the fact that the ratio of those whose lives were saved by life guards' use of the airway establishment instrument has increased drastically. This is because wounds are caused in varying ways, depending on accidents, and an inserting portion of the airway establishment instrument is inserted without visually inspecting where it is inserted into, and thus totally accurate operation thereof is difficult even though a user receives a training beforehand, so that it appears to be a reality that errors in emergency care, such as damaging the airway, taking too long a time to establish the airway and the like are not a few. The above-mentioned problem that the inserting portion of airway establishment instrument has to be inserted without visually inspecting where it is inserted into has occurred in the foreign body (e.g., phlegm or the like) removal instrument as well.

The present invention has been made to solve the above-mentioned problem, and therefore, it is an object of the invention to simplify the structure of a color image pickup and display device in which illuminating light is irradiated to a subject so that light reflected therefrom is picked up to thereby display the color image of a subject thus picked up on a display of a display device in real time, to thereby provide it at low price. It is another object of the invention to make a contribution to the small-sizing and price-reduction of devices that need to have a function to visually observe a narrow space, such as endoscopes, or devices that may desirably have an function to visually observe a narrow space, such as airway establishment instruments, foreign body removal instruments, microscopes and the like.

### SUMMARY OF THE INVENTION

According to a first aspect of the invention, there is provided a color image pickup and display device, including: a lighting means for irradiating light to a subject, the lighting means employing a lighting source that emits lights of different colors in a manner capable of switching the colors in a predetermined sequence; an image pickup means for picking up an image of the subject to be observed by reflection of light and outputting picture signals thereof, the image pickup means employing monochrome solid-state image sensors; a display device for receiving the picture signals and displaying the image of the subject on a display unit in real time, the display unit employing a backlight type monochrome liquid crystal display element capable of switching color of backlight to the same color as that of said lighting source synchronously with the switching of color by the lighting means, wherein the backlight is allowed to emit light of the same color as that of the light emitted by the lighting source when the picture signals outputted by the solid-state image sensors in response to the image observed by the light of the lighting source are displayed by the liquid crystal display element.

According to the first aspect of the invention, since the lighting means employs a lighting source that emits lights of different colors in a manner capable of switching the colors in a predetermined sequence, it is possible to obtain color picture signals even though the monochrome solid-state image sensors are used as an image pickup means, thus enabling the reduction of chip size of the sensors to about ⅓ or ¼ despite the same resolution.

Further, the color of the display backlight of the display device is switched to the same color as that of the lighting source synchronously with the switching of color by the lighting source, and thus it is possible to display and reproduce a color image of a subject to be observed, using the backlight type monochrome LCD element, thereby enabling the price reduction of the display unit of the display device.

Still further, since the monochrome display that is without a color filter for color separation is able to be used as LCD element, there would be no concern about the attenuation of light caused by the color filter, thus enabling the sensitivity to be enhanced. At the same time, the price of the display device can be reduced.

The color image pickup and display device according to a second aspect of the invention is the device of the first aspect, wherein the lighting source is provided outside a proximal side of an insertion tube of an endoscope, while light from the lighting source is allowed to be irradiated to a portion anterior to a distal side of the insertion tube, and wherein the solid-state image sensors are provided either outside a proximal portion of or at a distal side of the insertion tube of the endoscope so that light from an imaging lens provided at a distal side of the insertion tube is, either being guided by a light guiding means or directly, allowed to form image on surfaces of the solid-state image sensors, so as to be used as an endoscope.

According to the second aspect of the invention, the image of a portion anterior to the insertion body of the airway establishment instrument can be picked up in color, using the monochrome solid-state image sensors, and then reproduced in color by the display on the proximal side of the insertion body, and thus it is possible to insert the insertion body into the airway while visually grasping in what state a part anterior to the insertion body. Accordingly, the airway establishment using the airway establishment instrument can be performed much more easily than in the past, making life saving activities easier, thus eventually leading to the remarkable increase in survival rate.

The color image pickup and display device according to a third aspect of the invention is a device of the first aspect, wherein the lighting source is provided outside a proximal side of an insertion body of an airway establishment instrument, while light from the lighting source is allowed to be irradiated to a portion anterior to a distal side of the insertion body, and wherein the solid-state image sensors are provided either outside a proximal portion of or at a distal side of the insertion body of the airway establishment instrument so that light from an imaging lens provided at a distal side of the insertion body is, either being guided by a light guiding means or directly, allowed to form image on surfaces of the solid-state image sensors, so as to be used as an airway establishment instrument.

Accordingly, the airway establishment using the airway establishment instrument can be performed much more easily than in the past, making life saving activities easier, thus eventually leading to the remarkable increase in survival rate.

The color image pickup and display device according to a fourth aspect of the invention is a device of the first aspect, wherein the lighting source is provided outside a proximal side of an insertion body of a foreign body removal instrument, while light from the lighting source is allowed to be irradiated to a portion anterior to a distal side of the insertion body, and wherein the solid-state image sensors are provided either outside a proximal portion of or at a distal side of the insertion body so that light from an imaging lens provided at a distal side of the insertion body is, either being guided by a light guiding means or directly, allowed to form image on surfaces of the solid-state image sensors, so as to be used as a foreign body removal instrument.

According to the fourth aspect of the invention, the image of a portion anterior to the insertion body of the foreign body removal instrument can be picked up in color, using the monochrome solid-state image sensors, and then reproduced in color by the display on the proximal side of the insertion body, and thus it is possible to insert the insertion body into the airway while visually grasping in what state a part anterior to the insertion body. Accordingly, the removal of a foreign body (e.g., phlegm, rice cakes, gastric contents) using the foreign body removal instrument can be performed much more easily than in the past, making medical or emergency treatment easier, thus eventually leading to the ease of patient in receiving the treatment.

A color image pickup and display device according to a fifth aspect of the invention includes an image pickup means for picking up an image to be observed, the image pickup means sending picture signals to a display device in response to the image thus picked up to thereby display the image to be observed by a display unit thereof in real time, comprising:

a plurality of prisms each passing a specific different color component only;

a plurality of monochrome solid-state image sensors provided in response to the prisms, the monochrome solid-state image sensors picking up an image to be observed by light of the specific colors that have passed through the corresponding prisms, and then outputting picture signals to the display device in a manner capable of switching the outputs among the plurality of sensors,

wherein the display unit employs a backlight type monochrome liquid crystal display element capable of switching color of backlight to the same color as the light of the specific color that has passed through the prism synchronously with the switching of the outputs of the solid-state image sensors to the display device, and

wherein the backlight is allowed to emit light of the same color as that of the light picked up by the solid-state image sensors when one output from one of the solid-state image sensors is sent to the display device and thus the picture signals outputted by the solid-state image sensors are displayed by the liquid crystal display element.

According to a fifth aspect of the invention, there are provided a plurality of the prisms each passing a specific different color component only, and a plurality of the monochrome solid-state image sensors are provided in response to the prisms, so that the outputs of the monochrome solid-state image sensors to the display device are switched in a predetermined sequence, while the color of backlight of the display is switched synchronously with the switching of the outputs of the sensors, to thereby make the color of light received by the sensors consistent with the color of the backlight. As a result, it is not necessary to switch the color of the lighting. Thus, the invention can be applied to the color image pickup and display device which does not need lighting. In other words, the lighting source may be or may not be provided in the color image pickup and display device. If it is provided, however, the lighting of white color is preferable.

One embodiment of the invention is a case where the present invention is applied to a tubular inspection scope such as endoscope for visually inspecting a state inside a narrow and elongated space, in which a lighting source is arranged outside a proximal portion of an insertion tube so that light from the lighting source is guided by optical fibers disposed inside and along the insertion tube. In the case of airway establish instrument, optical fibers are disposed along the insertion body.

In a form of the invention, the (monochrome) solid-state image sensors may be arranged at a distal side of the insertion tube (or insertion body) and an imaging lens is disposed anterior to the solid-state image sensors.

In another form of the invention, the solid-state image sensors may be arranged outside the insertion tube (or insertion body). In that case, optical fiber group for guiding light from a subject to the proximal side of the insertion tube is provided inside or outside the insertion tube, while the imaging lens is provided on end faces of the distal side of the optical fibers so that the light of the image formed on the end faces is guided to the aforesaid solid-state image sensors to thereby pick up the image. The solid-state image sensors may be arranged according to either form of the above-mentioned.

In the meantime, the use of optical fibers is not essential for light guiding means, but as an other alternative means for guiding light from a light source may be used a plurality of mirrors disposed inside the tube to guide light by reflection of light.

The lighting source and the backlight may emit primary three colors: red, green and blue, but should not be limited thereto. For example, complementary colors such as cyan magenta yellow may be employed.

Further, complete color image pickup and color reproduction are not necessarily required. This is because esophagus or airway has a specific color (pink), and thus there are actually some cases that different color than white is rather suitable in order to visually inspect the inside thereof. If color imaging and reproduction are realized by the combination of two colors, then it will require only two lighting sources and backlights, respectively, thereby enabling the further small-sizing, light-weighting and price-reduction of the color image pickup and display device.

Preferably, the light emitting diodes (LED) may be used for the lighting source and the backlight, since LED is small and light, having a good luminous efficiency, and thus they are suitable for small-sizing and light-weighting of the device.

In the case where the present invention is applied to endoscopes, airway establishment instruments or foreign body (e.g., phlegm, rice cakes, gastric contents) removal instruments, an insertion tube (or insertion body) is necessary. In a form of the invention, a lighting source (e.g., LED), serving as a lighting means for lighting a distal side of the insertion tube (body), may be provided at a proximal side of the insertion tube (or body) so that light emitted from the lighting source is guided by light guiding means (e.g., optical fibers) to the distal of the insertion tube (or body) to light the distal side thereof. Alternatively, the lighting source such as LED may be provided at the distal side or in the vicinity of the tip end of the insertion tube (or body) to use the light therefrom as illumination light for directing lighting the distal portion.

In a further form of the invention, there may be provided a plurality of prisms each passing a specific different color component only, and a plurality of monochrome solid-state image sensors may be provided in response to the prisms, so that the outputs of the monochrome solid-state image sensors to the display device may be switched in a predetermined sequence, while the color of backlight of the display is switched synchronously with the switching of the outputs of the sensors, to thereby make the color of light received by the sensors consistent with the color of the backlight. In this case as well, the light passing through each prism may be in a primary color, or a complementary color. In the case of primary colors, complete color image pickup and color image reproduction are of course possible with the three primary colors: red, green and blue passing through the prisms. However, incomplete color image pickup and color image reproduction, which is realized with two colors, also may be applicable usefully enough, depending on cases.

In the above-mentioned case, there can be further two specific cases: one is a case where a lighting source is provided in the color image pickup and display device, while the other is a case where no lighting source is provided therein. In the case of the former, white light source may be preferably used as such lighting source, since white light contains all colors.

As discussed above, the present invention is applicable to endoscopes, airway establishment instruments and foreign body (e.g., phlegm, rice cakes, gastric contents, etc) removal instrument, but is also generally applicable to any color image pickup and display device of such a type that illuminating light is irradiated and then reflection is picked up so that an image thus picked up is displayed in color on a display of a display device in real time, in which display in color includes not only complete color display by the three primary colors, but may include incomplete color display by the two colors, or any other plural colors irrespective of whether they are primary or complementary. For example, the present invention may be applied to any insertion tube with scope function which is inserted into a small clearance or cavity of a building or a natural object to visually inspect the interior thereof.

It is to be noted that the present invention can be implemented in a case that a plurality of the prisms and the monochrome solid-state image sensors are provided but no lighting source are provided.

### BRIEF DESCRIPTION OF THE DRAWINGS

FIG. 1 is a schematic diagram showing a basic configuration of a color pickup and display device in accordance with an embodiment of the invention;

FIG. 2 is a diagram showing a principal part of a modified example of the color image pickup and display device of FIG. 1;

FIG. 3 is a perspective view showing an airway establishment instrument in accordance with the present invention; and

FIG. 4 is a schematic diagram showing a basic configuration of a color pickup and display device in accordance with another embodiment of the invention.

### DETAILED DESCRIPTION OF THE INVENTION

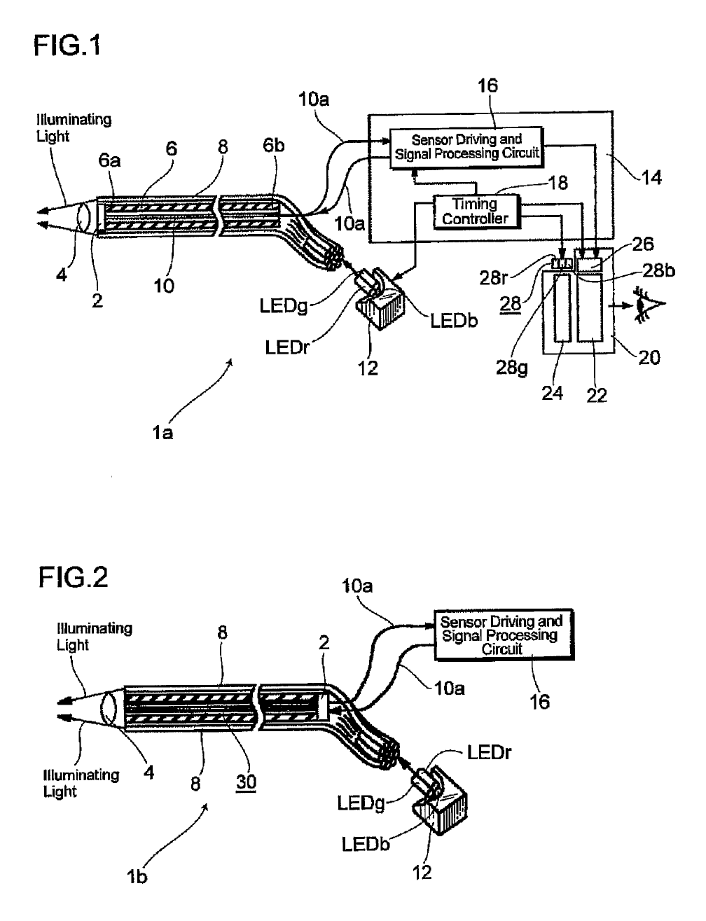

Next is a detailed description of the invention with reference to illustrated examples. FIG. 1 shows a basic configuration of a color pickup and display device 1a in accordance with a first example of the invention. Reference numeral 2 designates a solid-state image sensor, which is a monochrome solid-state image sensor. In other words, it is a solid-state image sensor without a color filter for color separation purpose, which may be of charge-transfer type (what is called CCD type), other amplifier type, MOS type or CMOS type. This solid-state image sensor is a monochrome solid-state image sensor, and the attenuation of light is decreased due to the absence of any color filter for color separation.

Reference numeral 4 designates an imaging lens provided on a distal side of the above-mentioned solid-state image sensor 2, said imaging lens 4 serving to focus a subject image on an image pickup surface of the solid-state image sensor 2. Reference numeral 6 designates an insertion tube of an endoscope or the like (corresponding to an inserting body in the case of an airway establishment instrument), in which 6a designates a distal side, while 6b a proximal side, and the above-mentioned solid-state image sensor is disposed at the distal side of the insertion tube 6. Reference numerals 8, 8 . . . designate optical fibers for lighting purpose, arranged outside the above-mentioned insertion tube 6 therealong, so that light from a hereinafter described light source 12 for lighting a subject is guided from the proximal side 6b toward the distal side 6a along the insertion tube 6 to thereby irradiate the distal side of the insertion tube 6 with the illuminating light. In the meantime, the outside of the optical fibers 8 are each covered with an outer sheath, which is not shown for the sake of simplicity.

Reference numeral 10 designates a group of wirings for connecting the solid-state image sensor 2 with an internal circuit 14, said group of wirings consisting of a wiring 10a for driving the solid-state image sensor 2 and a wiring for transmitting the picture signals outputted from the solid-state image sensor 2 to the internal circuit 14 described hereinafter. Reference numeral 12 designates a light source for lighting a subject, including three light emitting diodes of a red light emitting diode LEDr, a green light emitting diode LEDg and a blue light emitting diode LEDb so that the light emitting diode LED of the specific primary color corresponding to primary color switching signals from a hereinafter-described timing controller 18 is lighted. The light source 12 allows the light of the respective primary colors from the light emitting diodes LEDr, g, and b to be outputted to proximal end faces of the group of the optical fibers 8.

Reference numeral 14 designates the internal circuit, comprising a sensor driving and signal processing circuit 16 and the timing controller 18. The sensor driving and signal processing circuit 16 outputs various driving signals to the solid-state image sensor 2, and receives the picture signals outputted from the solid-state image sensor 2, allows them to undergo amplification processing or the like, and then sends thus processed signals to a hereinafter-described liquid crystal display (LCD) element drive circuit 26 of a display device 20.

It should be noted that the sensor driving and signal processing circuit 16 need not generate color television picture signals (for instance, picture signals according to NTSC, PAL or SECAM system), and thus circuit configuration thereof may be simple, even if it might be necessary for the signals from the solid-state image sensor 2 to undergo simple processing such as amplification, rectification or timing adjustment required due to delay. Thus, the sensor driving and signal processing circuit 16 can be provided at a low price.

The timing controller 18 outputs drive-timing controlling signals to the aforesaid light source 12, the sensor driving and signal processing circuit 16 and a hereinafter-described backlighting light source 28. In the meantime, the action of the timing controller 18 will be described in detail later.

Reference numeral 20 designates a display device, including a backlight LCD element 22 as a main component thereof. The backlight LCD element is of a monochrome type, including no color filter for color separation. Thus, there is no concern about the attenuation of display light being caused by the color filter.

Reference numeral 24 designates a backlight of the backlight LCD element, receiving light from the backlighting light source 28 and eliminating the backlight LCD element 22 from behind. Reference numeral 26 designates an LCD element drive circuit, processing the picture signals from the sensor driving and signal processing circuit 16 and then sending the signals thus processed to the LCD element 22, while the timing in the processing of the picture signals being controlled by the timing controller 18.

Reference numeral 28 designates the backlighting light source, comprising three light emitting means 28r, 28g, and 28b (e.g., light emitting diodes) to emit light of three different primary colors: red, green and blue so that the light emitting means (such as diode) 28r, 28g or 28b of the specific primary color may be eliminated in response to light source color switching signals from the timing controller 18.

Next is a detailed description of the action of the above-mentioned timing controller 18. For example, the red light emitting diode LEDr begins lighting on a certain timing, while start signals are sent to the sensor driving and signal processing circuit 16 in order for the solid-state image sensor 2 to start the pickup of one-field image synchronously therewith. The sensor driving and signal processing circuit 16 receives the start signals and then allows the solid-state image sensor 2 to start the pickup of one-field image in 1/180 second.

On the other and, the timing controller 18 sends signals for lighting the red light emitting means 28r to the backlighting light source 28 with the precise timing when the aforesaid one-field picture signals begin to come from the solid-state image sensor 2, so that the red backlight is irradiated from the backlight 24, while sending signals for displaying the one-field image to the aforesaid LCD element 22. Accordingly, the LCD element 22 is brought into a state displaying the red signal of a subject.

The image pickup and display of a subject by the red light lasts 1/180 second. This is an operation in the red field.

When the image pickup and display of the red field are finished, then the pickup and display of a green field are carried out in totally the same manner. To summarize it, the diode LED for lighting a subject lighting source 12 is switched to the green light emitting diode LEDg, while that of the backlighting light source 28 is switched to the green light emitting diode 28b, and then the pickup and display is carried out. The image pickup and display in the green filed also lasts 1/180 second.

Subsequently, when the image pickup and display of the green field are finished, then the pickup and display of a blue field are carried out in totally the same manner. That is, the diode LED for lighting a subject lighting source 12 is switched to the blue light emitting diode LEDb, while that of the backlighting light source 28 is switched to the blue light emitting diode 28b, and then the pickup and display is carried out. The image pickup and display in the blue filed also lasts 1/180 second.

With the operation in the red, green and blue fields, i.e., the operation of 1/60 second being set as one cycle, the operation of displaying respective parts of a subject with the color thereof is repeated per each pixel during the operation of the color image pickup and display device 1a.

According to the color image pickup and display device 1a, therefore, it is possible for each pixel of the solid-state image sensor 2 to pick up the color image of the part of a subject corresponding to each pixel, and to display it by the monochrome LCD element 22.

Therefore, pickup of color image is possible using the solid-state image sensor 2 that is smaller than conventional ones but has the same resolution as them. Further, color picture can be displayed by the monochrome LCD element 22. As a result, neither the solid-state image sensor 2 nor the monochrome LCD element 22 needs the color filter for color separation, which in turn means that the attenuation of light can be avoided in the two stages: picking up images and displaying the images, and that the solid-state image sensor 2 as well as the monochrome LCD element 22 can be provided at a low price. Eventually, this means that resolution can be enhanced up to three to four times in the case that the solid-state image sensor 2 of the same chip size is used.

### MODIFIED EXAMPLE

FIG. 2 is a schematic diagram showing a principal part of another embodiment 1b of the color image pickup and display device 1a. The device 1b differs from the former in that the solid-state image sensor 2 is not provided in the distal side 6a of the insertion tube (which corresponds to the insertion body in the case of airway establishment instrument) but in the proximal portion 6b thereof.

Optical fiber group 30 (the number of fibers being for instance about the number of pixels of the solid-state image sensor 2) consisting of a lot of optical fibers is provided inside the insertion tube 6, one edge surface thereof is exposed to the distal end surface of the insertion tube 6, while the other edge surface thereof is disposed opposite to an image pickup surface of the solid-state image sensor 2.

The imaging lens 4 for forming the image of a subject is arranged so as to form the image on an exposed distal end surface of the optical fiber group 30 at the distal end of the insertion tube 6 thereof. The light on the respective parts of a subject focused on the end surface of the optical fiber group 30 is guided by each optical fiber of the optical fiber group 30 to reach the image pickup surface of the solid-state image sensor 2. Thus, the image of a subject is formed virtually directly on the image pickup surface of the solid-state image sensor 2. In the meantime, the color image pickup and display device 1b according to the present modified example has the same configuration as the aforesaid device 1a of FIG. 1 except the above-mentioned feature.

The respective optical fibers of the optical fiber group 30 need to be precisely positioned so as not to include a positional error between the distal side and the proximal side. Otherwise, the image of a subject formed by the imaging lens on the distal end surface of the optical fiber group 30 will not reach the surface of the solid-state image sensor 2 as it is, but a distorted image will reach there instead. If the number of the optical fibers that make up the optical fiber group 30 is increased up to about the same as the number of pixels of the solid-state image sensor 2, then it would improve the image quality but become expensive.

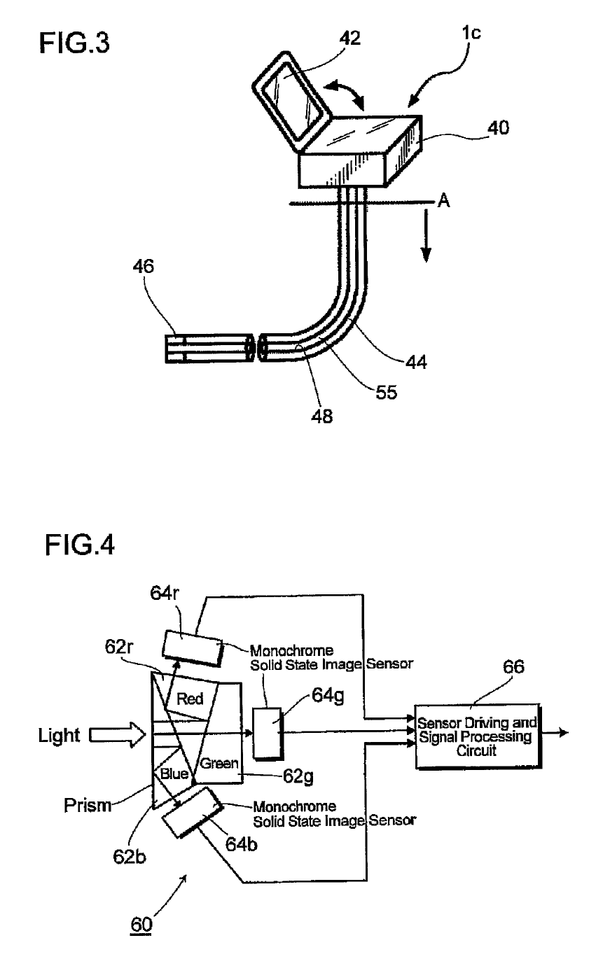

FIG. 3 is a perspective view of another example 1c in which the present invention is applied to an airway establishment instrument. Reference numeral designates an instrument body, incorporating various circuits, a solid-state image sensor and the like, to which is attached a backlight type LCD element 42 in an openable and closable manner. Reference numeral 44 designates an insertion body of a tube shape, having a proximal portion fixed to a bottom surface of the instrument body 40. Reference numeral 46 designates a tip end of the insertion body 44, including a built-in imaging lens (not shown), outputting illuminating light forward.

The airway establishment instrument 1c allows a distal area thereof to be monitored visually with the LCD display element 42 according to the same mechanism and the principle as described in connection with the color image pickup and display device 1a or 1c shown in FIG. 1 or 2.

Accordingly, when an emergency life guard inserts a tube 48 attached to a groove 55 of an outer side surface of the insertion body 44 into a human airway to establish his airway, the tube 48 attached to the insertion body 44 can be advanced into the airway while visually grasping in what state a part anterior to the insertion body 44 is situated, thus enabling the emergency life guard to smoothly and speedily establish the airway. Thus, it is possible to noticeably decrease errors in the air establishment and the life savings.

In the meantime, since the insertion body 44 is contaminated with bacillus etc. from patient's saliva or the like once it is used, a disposable type that is replaceable at the proximal position (as shown in line A of FIG. 3) has been developed, as proposed in Japanese Un-Examined Patent Publication No. 2004-280528. In that case, the solid-state image sensor may be provided on the proximal side of the insertion body 44 (or inside the device body 40) and a light guiding means utilizing mirror may be provided instead of the optical fiber group, in order to make the price of the insertion body the lowest possible. Namely, a plurality of mirrors may be arranged inside the tubular insertion body so that the formed image of a subject may be transmitted to the solid-state image sensor at the proximal side of the insertion body.

FIG. 4 is a schematic diagram showing a principal part of a further embodiment 60 of the color image pickup and display device of the invention.

Reference numeral 62r designates a prism through which red light only passes, 62g a prism through which green light only passes, and 62b a prism through which blue light only passes, respectively, which are arranged so as to face an image to be observed via the imaging lens (not shown).

Reference numeral 64r designates a monochrome solid-state image sensor provided corresponding to the red color prism 62r so that it may pick up the image of a subject composed of red color only that has passed therethrough, 64g a monochrome solid-state image sensor provided corresponding to the green color prism 62g so that it may pick up the image of a subject composed of green color only that has passed therethrough, and 64b a monochrome solid-state image sensor provided corresponding to the blue color prism 62b so that it may pick up the image of a subject composed of blue color only that has passed therethrough, respectively.

Reference numeral 66 designates a sensor driving and signal processing circuit, outputting respective signals for driving the respective monochrome solid-state image sensors 64r, 64g and 64b, also functioning to receive the picture signals from the respective monochrome solid-state image sensors 64r, 64g and 64b and then to send the output signals therefrom to the display device (not shown but is the same as the display device 20 of FIG. 1) in a predetermined sequence.

Further, the sensor driving and signal processing circuit 66 functions to send signals for switching the color of light of a light source to the light source of a backlight of a display unit of the display device, in which the light source is the same as the backlighting source 28 of FIG. 1, the backlight is the same as the backlight 24 of FIG. 1, and the display unit is the same as that of FIG. 1, being composed of the LCD element 22.

Next is a description of the behavior of the color image pickup and display device.

The above-mentioned monochrome solid-state image sensors 64r, 64g and 64b are allowed to perform image pickup actions synchronously with one another. In other words, the sensor 64r takes a red color image, 64g a green color image, and 64b a blue color image, respectively and parallel to one another.

The sensor driving and signal processing circuit 66 receives picture output signals from the three monochrome solid-state image sensors 64r, 64g and 64b, and then send processed picture signals to the aforesaid display device in a predetermined sequence, such as 64r→64g→64b→64r→64g→64b.

Further, the sensor driving and signal processing circuit 66 functions to send color switching signals for generating red light in the backlighting source when the output of the red-color-image taking solid-state image sensor 64r is sent to the display device, those for generating green light in the backlighting source when the output of the green-color-image taking solid-state image sensor 64g is sent to the display device, and those for generating red light in the backlighting source when the output of the blue-color-image taking solid-state image sensor 64b is sent to the display device, respectively.

In other words, the color which each monochrome solid-state image sensor sending the output to the display device receives is arranged so as to be always the same as the color of the backlight.

Accordingly, it is possible to realize complete color image pickup and color image reproduction.

In the meantime, the color image pickup and display device of the present invention does not always need the lighting source. In an alternative form of the invention, therefore, the monochrome solid-state image sensors 64r, 64g and 64b may receive light from an image to be observed via the prisms 62r, 62g and 62b and then output the picture signals.

Needless to say, in order for the color image pickup and display device of the present invention to be able to be used for observation of such a dark portion that necessitates illumination, the color image pickup and display device may be provided with the lighting source for illuminating such portion to be observed. In that case, white light source is preferably used as such lighting source, since white light contains all colors. Alternatively, the prisms are not limited to ones passing primary colors, but may be ones passing complementary colors.

Alternatively, the number of the prisms may be two in the example of FIG. 4 such that the monochrome solid-state image sensor is provided in response to each prism, to thereby perform incomplete color image pickup and incomplete color image reproduction. In some cases that the color image pickup and reproduction do not have to be complete, even such color image pickup and display device is useful enough, and thus the number of the prisms and solid-state image sensors can be reduced, thus contributing to the small-sizing, light-weighting and price-reduction of the color image pickup and display device

In the meantime, the present invention is not only applicable to endoscopes, airway establishment instruments and foreign body removal instruments, as discussed above, but is also generally applicable to any color image pickup and display device of such a type that illuminating light is irradiated and then reflection is picked up so that an image thus picked up is displayed in color on a display of a display device in real time. For example, the present invention may be applied to any insertion tube with scope function which is inserted into a small clearance or cavity of a building or a natural object to visually inspect the interior.

1. A color image pickup and display device, comprising:
a lighting means for irradiating light to a subject, said lighting means employing a lighting source that emits lights of different colors in a manner capable of switching the colors in a predetermined sequence;
an image pickup means for picking up an image of the subject to be observed by reflection of light and outputting picture signals thereof, said image pickup means employing monochrome solid-state image sensors;
a display device for receiving the picture signals and displaying the image of the subject on a display unit in real time, said display unit employing a backlight type monochrome liquid crystal display element capable of switching color of backlight to the same color as that of said lighting source synchronously with the switching of color by said lighting means,
wherein said backlight is allowed to emit light of the same color as that of the light emitted by the lighting source when the picture signals outputted by said solid-state image sensors in response to the image observed by the light of said lighting source are displayed by said liquid crystal display element.

2. The color image pickup and display device according to claim 1, wherein lights of different colors emitted by said lighting source and said backlight are primary colors consisting of red, blue and green.

3. The color image pickup and display device according to claim 1, wherein lights of different colors emitted by said lighting source and said backlight are complementary colors.

4. The color image pickup and display device according to claim 1, wherein lights of different colors emitted by said lighting source and said backlight are of two colors.

5. The color image pickup and display device according to claim 1, wherein said lighting means comprises a plurality of light emitting diodes emitting lights of different colors, lighting of said light emitting diodes being switched in a predetermined sequence.

6. The color image pickup and display device according to claim 2, wherein said lighting means comprises three light emitting diodes emitting lights of different primary colors, lighting of said light emitting diodes being switched in a predetermined sequence.

7. The color image pickup and display device according to claim 1,
wherein said lighting source is provided outside a proximal side of an insertion tube of an endoscope, while light from said lighting source is allowed to be irradiated to a portion anterior to a distal side of said insertion tube, and
wherein said solid-state image sensors are provided either outside a proximal portion of or at a distal side of the insertion tube of the endoscope so that light from an imaging lens provided at a distal side of the insertion tube is, either being guided by a light guiding means or directly, allowed to form image on surfaces of said solid-state image sensors, so as to be used as an endoscope.

8. The color image pickup and display device according to claim 7, wherein lights of different colors emitted by said lighting source and said backlight are primary colors consisting of red, blue and green.

9. The color image pickup and display device according to claim 7, wherein lights of different colors emitted by said lighting source and said backlight are complementary colors.

10. The color image pickup and display device according to claim 7, wherein lights of different colors emitted by said lighting source and said backlight are of two colors.

11. The color image pickup and display device according to claim 7, wherein said lighting means comprises a plurality of light emitting diodes emitting lights of different colors, lighting of said light emitting diodes being switched in a predetermined sequence.

12. The color image pickup and display device according to claim 8, wherein said lighting means comprises three light emitting diodes emitting lights of different primary colors, lighting of said light emitting diodes being switched in a predetermined sequence.

13. The color image pickup and display device according to claim 1,
wherein said lighting source is provided outside a proximal side of an insertion body of an airway establishment instrument, while light from said lighting source is allowed to be irradiated to a portion anterior to a distal side of said insertion body, and
wherein said solid-state image sensors are provided either outside a proximal portion of or at a distal side of the insertion body of the airway establishment instrument so that light from an imaging lens provided at a distal side of the insertion body is, either being guided by a light guiding means or directly, allowed to form image on surfaces of said solid-state image sensors, so as to be used as an airway establishment instrument.

14. The color image pickup and display device according to claim 13, wherein lights of difference colors emitted by said lighting source and said backlight are primary colors consisting of red, blue and green.

15. The color image pickup and display device according to claim 13, wherein said lighting means comprises a plurality of light emitting diodes emitting lights of different colors, lighting of said light emitting diodes being switched in a predetermined sequence.

16. The color image pickup and display device according to claim 14, wherein said lighting means comprises three light emitting diodes emitting lights of different primary colors, lighting of said light emitting diodes being switched in a predetermined sequence.

17. The color image pickup and display device according to claim 1,
wherein said lighting source is provided outside a proximal side of an insertion body of a foreign body removal instrument, while light from said lighting source is allowed to be irradiated to a portion anterior to a distal side of said insertion body, and
wherein said solid-state image sensors are provided either outside a proximal portion of or at a distal side of said insertion body so that light from an imaging lens provided at a distal side of the insertion body is, either being guided by a light guiding means or directly, allowed to form image on surfaces of said solid-state image sensors, so as to be used as a foreign body removal instrument.

18. The color image pickup and display device according to claim 17, wherein lights of different colors emitted by said lighting source and said backlight are primary colors consisting of red, blue and green.

19. The color image pickup and display device according to claim 17, wherein said lighting means comprises a plurality of light emitting diodes emitting lights of different colors, lighting of said light emitting diodes being switched in a predetermined sequence.

20. The color image pickup and display device according to claim 18, wherein said lighting means comprises three light emitting diodes emitting lights of different primary colors, lighting of said light emitting diodes being switched in a predetermined sequence.

21. The color image pickup and display device according to claim 1, wherein said color image pickup and display device is used as a microscope.

22. The color image pickup and display device according to claim 1,
wherein said lighting source is provided outside a proximal side of an insertion body that is inserted into a portion to be imaged, while said solid-state image sensors are provided either outside a proximal portion of or at a distal side of said insertion body so that light from an imaging lens provided at a distal side of the insertion body is, either being guided by a light guiding means or directly, allowed to form image on surfaces of said solid-state image sensors.

23. A color image pickup and display device, including an image pickup means for picking up an image to be observed, said image pickup means sending picture signals to a display device in response to the image thus picked up to thereby display the image to be observed by a display unit thereof in real time, comprising:
a plurality of prisms each passing a specific different color component only;
a plurality of monochrome solid-state image sensors provided in response to said prisms, said monochrome solid-state image sensors picking up an image to be observed by light of the specific colors that have passed through the corresponding prisms, and then outputting picture signals to said display device in a manner capable of switching the outputs among the plurality of sensors,
wherein said display unit employs a backlight type monochrome liquid crystal display element capable of switching color of backlight to the same color as the light of said specific color that has passed through said prism synchronously with the switching of the outputs of said solid-state image sensors to said display device, and
wherein said backlight is allowed to emit light of the same color as that of the light picked up by said solid-state image sensors when one output from one of said solid-state image sensors is sent to said display device and thus the picture signals outputted by said solid-state image sensors are displayed by said liquid crystal display element.

24. The color image pickup and display device according to claim 23, wherein the number of said prisms and the number of said solid-state image sensors are each three, each prism passing through a specific primary color of red, blue or green different from the primary colors the other prisms pass.
